(19) United States
(12) Patent Application Publication
(10) Pub. No.: US 2020/0230136 A1
(43) Pub. Date: Jul. 23, 2020

(54) SELECTIVE ADRENORECEPTOR ALPHA2C RECEPTOR ANTAGONISTS ALONE, OR IN COMBINATION WITH CHYMASE INHIBITORS FOR USE IN THE TREATMENT AND/OR PROPHYLAXIS OF PERIPHERAL ARTERY DISEASES (PAD)

(71) Applicant: Bayer Aktiengesellschaft, Leverkusen (DE)

(72) Inventors: Eva Maria BECKER-PELSTER, Wuppertal (DE); Christiane OTTO, Wuppertal (DE); Carsten SCHMECK, Mülheim (DE); Hanna TINEL, Wuppertal (DE); Chantal FÜRSTNER, Mülheim (DE)

(73) Assignee: Bayer Aktiengesellschaft, Leverkusen (DE)

(21) Appl. No.: 16/608,710

(22) PCT Filed: Apr. 19, 2018
(86) PCT No.: PCT/EP2018/060053

§ 371 Date: Oct. 25, 2019

(30) Foreign Application Priority Data
Apr. 27, 2017 (EP) 17168562.1

(51) Int. Cl. A61K 31/506; A61K 31/541; A61K 31/5377; A61P 9/10

(52) U.S. Cl. CPC A61K 31/506; A61P 9/10; A61K 31/5377; A61K 31/541

(57) ABSTRACT

The invention relates to selective adrenoreceptor α_{2C} receptor antagonists alone, or in combination with chymase inhibitors for use in the treatment and/or prophylaxis of peripheral artery diseases (PAD) and/or critical limb ischemia (CLI).

The invention relates to selective adrenoreceptor α_{2C} receptor antagonists alone, or in combination with chymase inhibitors for use in the treatment and/or prophylaxis of peripheral artery diseases (PAD) and/or critical limb ischemia (CLI).

Peripheral artery disease (PAD) can have various clinical presentations (Semin Intervent Radiol 2014, 31: 378-388) and is divided into 4 subgroups:

asymptomatic,

intermittent claudication,

acute limb ischemia,

critical limb ischemia (CLI).

CLI as most severe subgroup of PAD is characterized by the presence of rest pain in the legs, leg ulcers and/or gangrene. Patients suffering from CLI have a high medical need. They suffer from reduced macro- and microperfusion to the limb and the skeletal muscles, quite often in combination with comorbidities such as atherosclerosis, hypertension and diabetes. CLI patients are suffering as PAD patients in general from a reduced walking distance due to limited blood flow to the lower extremities. Reduced perfusion in capillaries of skeletal muscles reduces oxygen supply and impairs disposal of metabolic endproducts causing pain in the skeletal musculature that forces the patient to stop walking

PAD is thought to be primarily a disease of the macrovasculature that is affected by atherosclerosis. Impaired blood flow in the macrovasculature can be addressed by venous bypass grafting, stent implantation or balloon dilatation. However, there is a high risk of restenosis due to adverse vascular remodeling and underlying atherosclerosis.

In general, symptoms such as intermittent claudication or non-healing leg ulcers in CLI patients are not observed as long as capillary blood flow in the skin and skeletal musculature allows for sufficient oxygen supply and disposal of metabolites.

Accordingly, it is an object of the present invention to provide suitable compounds for the use in the treatment and/or prophylaxis of peripheral artery diseases (PAD) and/or critical limb ischemia (CLI) diseases and to improve the macro- and microcirculation of patients suffering from PAD and/or CLI.

It was found that a combination of selective adrenoreceptor α_{2C} receptor antagonists and at least one chymase inhibitor represents a meaningful treatment for PAD/CLI patients by improving blood flow in the impaired macro- and microcirculation.

WO2015091414 and WO2015091417 describe substituted piperidinyltetrahydroquinolines acting as selective adrenoreceptor α_{2C} receptor antagonists for use in the treatment and/or prophylaxis of diseases such as, for example, cardiovascular disorders, in humans and animals and of peripheral circulatory disturbances (microangiopathies) such as, for example, diabetic retinopathy, diabetic nephropathy and wound healing disorders (diabetic foot ulcers).

The invention provides selective adrenoreceptor α_{2C} receptor antagonists of the formula (I)

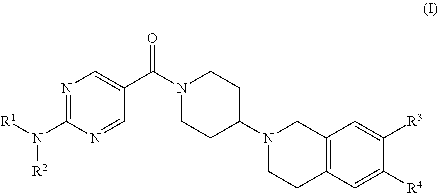

in which

R¹ represents C₁-C₆-alkyl or C₃-C₅-cycloalkyl,
where alkyl is substituted by 1 to 2 substituents independently of one another selected from the group consisting of hydroxy and C₁-C₄-alkoxy

and

R² represents hydrogen or C₁-C₄-alkyl,

or

R¹ and R² together with the nitrogen atom to which they are attached form a 4- to 7-membered N-heterocycle,
where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy and halogen,
or
where the N-heterocycle may have two substituents which, together with the carbon atom of the N-heterocycle to which they are jointly attached, form a 4- to 6-membered heterocycle,
   where this heterocycle for its part may be substituted by 1 to 3 substituents independently of one another selected from the group consisting of oxo, methyl and ethyl,

R³ represents hydrogen, fluorine, methoxy or ethoxy,

and

R⁴ represents hydrogen, fluorine, methoxy or ethoxy,

and the salts thereof, the solvates thereof and the solvates of the salts thereof,

for use in the treatment and/or prophylaxis of peripheral artery diseases (PAD) and/or critical limb ischemia (CLI).

The compounds of formule (I) and the synthesis thereof are known from WO2015091414.

Preference is given to compounds of the formula (I) in which

R¹ represents C₂-C₆-alkyl,
where alkyl is substituted by a substituent selected from the group consisting of hydroxy, methoxy and ethoxy,

and

R² represents hydrogen or

R¹ and R² together with the nitrogen atom to which they are attached form an azetidine, pyrrolidine, piperidine, azepane, piperazine, morpholine, thiomorpholine, 1-oxidothiomorpholine or 1,1-dioxidothiomorpholine,
where azetidine, pyrrolidine, piperidine, azepane, piperazine, morpholine, thiomorpholine, 1-oxidothiomorpholine and 1,1-dioxidothiomorpholine may be substituted by 1 to 2 substituents independently of one another selected from the group consisting of hydroxy, trifluoromethyl, hydroxycarbonyl, C₁-C₃-alkyl, methoxy and methoxymethyl,
or
where azetidine, pyrrolidine, piperidine, azepane, piperazine and morpholine may have two substituents which, together with the carbon atom of the azetidine, pyrrolidine, piperidine, azepane, piperazine or morpholine to which they are jointly attached, form an azetidine, oxetane or 1,1-dioxidothietane,
   where this azetidine, oxetane or 1,1-dioxidothietane for its part may be substituted by 1 to 2 substituents independently of one another selected from the group consisting of methyl and ethyl,

R³ represents hydrogen,

and

R⁴ represents hydrogen, fluorine or methoxy

or

R³ represents hydrogen, fluorine or methoxy

and

R⁴ represents hydrogen,

and the salts thereof, the solvates thereof and the solvates of the salts thereof.

Preference is given to compounds of the formula (I) in which

R¹ represents C₂-C₄-alkyl,
where alkyl is substituted by a substituent selected from the group consisting of hydroxy and methoxy,

and

R² represents hydrogen,

or

R¹ and R² together with the nitrogen atom to which they are attached form an azetidine, pyrrolidine, morpholine or 1,1-dioxidothiomorpholine,
where azetidine, pyrrolidine, morpholine or 1,1-dioxidothiomorpholine may be substituted by 1 to 2 substituents selected independently from the group consisting of hydroxycarbonyl, methyl, trifluoromethyl, methoxy and methoxymethyl,
or

R¹ and R² together with the nitrogen atom to which they are attached form an azetidine,
where the azetidine may have two substituents which, together with the carbon atom of the azetidine to which they are jointly attached, form an oxetane or 1,1-dioxidothietane,

R³ represents hydrogen, fluorine or methoxy

and

R⁴ represents hydrogen,

or

R³ represents hydrogen,

and

R⁴ represents hydrogen, fluorine or methoxy

and the salts thereof, the solvates thereof and the solvates of the salts thereof.

Preference is given to compounds of the formula (I) in which

R¹ represents C₂-C₄-alkyl,
where alkyl is substituted by a substituent selected from the group consisting of hydroxy and methoxy,

and

R² represents hydrogen,

or

R¹ and R² together with the nitrogen atom to which they are attached form an azetidine, pyrrolidine, morpholine or 1,1-dioxidothiomorpholine,
where azetidine, pyrrolidine, morpholine or 1,1-dioxidothiomorpholine may be substituted by 1 to 2 substituents selected independently from the group consisting of hydroxycarbonyl and methyl,

or

R¹ and R² together with the nitrogen atom to which they are attached form an azetidine,
where the azetidine may have two substituents which, together with the carbon atom of the azetidine to which they are jointly attached, form an oxetane,

R³ represents hydrogen,

and

R⁴ represents hydrogen,

and the salts thereof, the solvates thereof and the solvates of the salts thereof.

Preference is given to compounds of the formula (I) in which

R¹ and R² together with the nitrogen atom to which they are attached form an azetidine,
where the azetidine has two substituents which, together with the carbon atom of the azetidine to which they are jointly attached, form an oxetane,

R³ represents hydrogen,

and

R⁴ represents hydrogen,

and the salts thereof, the solvates thereof and the solvates of the salts thereof.

Preference is given to compounds of the formula (I) in which

R¹ represents C₁-C₆-alkyl,
where alkyl is substituted by 1 to 2 substituents independently of one another selected from the group consisting of hydroxy, C₁-C₄-alkoxy and cycloalkyloxy

and

R² represents hydrogen or C₁-C₄-alkyl,

or

R¹ and R² together with the nitrogen atom to which they are attached form a 4- to 7-membered N-heterocycle,
where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy and halogen,
or
where the N-heterocycle may have two substituents which, together with the carbon atom of the N-heterocycle to which they are jointly attached, form a 4- to 6-membered heterocycle,
   where this heterocycle for its part may be substituted by 1 to 3 substituents independently of one another selected from the group consisting of oxo, methyl and ethyl,

R³ represents hydrogen, fluorine, methoxy or ethoxy,

and

R⁴ represents hydrogen, fluorine, methoxy or ethoxy,

and the salts thereof, the solvates thereof and the solvates of the salts thereof.

Preference is given to compounds of the formula (I) in which

R¹ represents C₂-C₆-alkyl,
where alkyl is substituted by a substituent selected from the group consisting of hydroxy, methoxy and ethoxy,

and

R² represents hydrogen,

or

R¹ and R² together with the nitrogen atom to which they are attached form an azetidine, pyrrolidine, piperidine, azepane, piperazine, morpholine, thiomorpholine, 1-oxidothiomorpholine or 1,1-dioxidothiomorpholine,
where azetidine, pyrrolidine, piperidine, azepane, piperazine, morpholine, thiomorpholine, 1-oxidothiomorpholine and 1,1-dioxidothiomorpholine may be substituted by 1 to 2 substituents independently of one another selected from the group consisting of hydroxy, hydroxycarbonyl, C₁-C₃-alkyl and methoxy,
or
where azetidine, pyrrolidine, piperidine, azepane, piperazine and morpholine may have two substituents which, together with the carbon atom of the azetidine, pyrrolidine, piperidine, azepane, piperazine or morpholine to which they are jointly attached, form an azetidine or oxetane,
   where this azetidine or oxetane for its part may be substituted by 1 to 2 substituents independently of one another selected from the group consisting of methyl and ethyl,

R³ represents hydrogen,

and

R⁴ represents hydrogen, fluorine or methoxy

or

R³ represents hydrogen, fluorine or methoxy

and

R⁴ represents hydrogen,

and the salts thereof, the solvates thereof and the solvates of the salts thereof.

Preference is also given to compounds of the formula (I) in which

R¹ represents C₂-C₆-alkyl,
where alkyl is substituted by a substituent selected from the group consisting of hydroxy, methoxy and ethoxy,

and

R² represents hydrogen,

or

R¹ and R² together with the nitrogen atom to which they are attached form an azetidine, pyrrolidine, piperidine, azepane, piperazine, morpholine, thiomorpholine, 1-oxidothiomorpholine or 1,1-dioxidothiomorpholine,
where azetidine, pyrrolidine, piperidine, azepane, piperazine, morpholine, thiomorpholine, 1-oxidothiomorpholine and 1,1-dioxidothiomorpholine may be substituted by 1 to 2 substituents independently of one another selected from the group consisting of hydroxy, hydroxycarbonyl, C₁-C₃-alkyl and methoxy,
or
where azetidine, pyrrolidine, piperidine and azepane may have two substituents which, together with the carbon atom of the azetidine, pyrrolidine, piperidine or azepane to which they are jointly attached, form an azetidine or oxetane,
   where this azetidine or oxetane for its part may be substituted by 1 to 2 substituents independently of one another selected from the group consisting of methyl and ethyl,

R³ represents hydrogen,

and

R⁴ represents hydrogen, fluorine or methoxy

or

R³ represents hydrogen, fluorine or methoxy

and

R⁴ represents hydrogen,

and the salts thereof, the solvates thereof and the solvates of the salts thereof.

Preference is also given to compounds of the formula (I) in which

R¹ represents C₂-C₄-alkyl,
where alkyl is substituted by a substituent selected from the group consisting of hydroxy and methoxy,

and

R² represents hydrogen,

or

R¹ and R² together with the nitrogen atom to which they are attached form an azetidine, pyrrolidine, morpholine or 1,1-dioxidothiomorpholine,
where azetidine, pyrrolidine, morpholine or 1,1-dioxidothiomorpholine may be substituted by 1 to 2 substituents selected independently from the group consisting of oxo, hydroxy, hydroxycarbonyl and methyl,

or

R¹ and R² together with the nitrogen atom to which they are attached form an azetidine,
where the azetidine may have two substituents which, together with the carbon atom of the azetidine to which they are jointly attached, form an oxetane,

R³ represents hydrogen,

and

R⁴ represents hydrogen,

and the salts thereof, the solvates thereof and the solvates of the salts thereof.

Preference is also given to compounds of the formula (I) in which

R¹ represents C₂-C₆-alkyl,
where alkyl is substituted by a substituent selected from the group consisting of hydroxy, methoxy and ethoxy,

and

R² represents hydrogen,

and the salts thereof, the solvates thereof and the solvates of the salts thereof.

Preference is also given to compounds of the formula (I) in which

R¹ and R² together with the nitrogen atom to which they are attached form an azetidine, pyrrolidine, morpholine or 1,1-dioxidothiomorpholine,
where azetidine, pyrrolidine, morpholine or 1,1-dioxidothiomorpholine may be substituted by 1 to 2 substituents selected independently from the group consisting of oxo, hydroxy, hydroxycarbonyl and methyl,

or

R¹ and R² together with the nitrogen atom to which they are attached form an azetidine,
where the azetidine may have two substituents which, together with the carbon atom of the azetidine to which they are jointly attached, form an oxetane,

and the salts thereof, the solvates thereof and the solvates of the salts thereof.

Preference is also given to compounds of the formula (I) in which R² represents hydrogen.

Preference is also given to compounds of the formula (I) in which R¹ and R² together with the nitrogen atom to which they are attached represent 2-oxa-6-azaspiro[3.3]hept-6-yl.

Preference is also given to compounds of the formula (I) in which R¹ and R² together with the nitrogen atom to which they are attached represent 1,1-dioxidothiomorpholin-4-yl.

Preference is also given to compounds of the formula (I) in which R³ represents hydrogen.

Preference is also given to compounds of the formula (I) in which R⁴ represents hydrogen.

Preference is also given to compounds of the formula (I) in which R³ and R⁴ represent hydrogen.

The individual radical definitions specified in the particular combinations or preferred combinations of radicals are, independently of the particular combinations of the radicals specified, also replaced as desired by radical definitions of other combinations.

Very particular preference is given to combinations of two or more of the abovementioned preferred ranges.

According to a further embodiment, the compounds of formula (I) for use in treatment of peripheral artery diseases (PAD) and/or CLI are selected from the working examples of WO2015091414:

### EXAMPLE1

[4-(3,4-Dihydroisoquinolin-2(1H)-yl)piperidin-1-yl]{2-[(2-methoxyethyl)amino]pyrimidin-5-yl}methanone of the formula

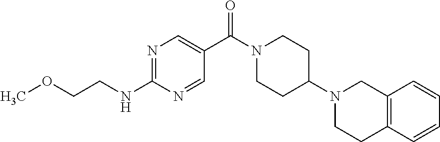 [SMILES: COCCNc1ncc(C(=O)N2CCC(N3CCc4ccccc4C3)CC2)cn1]

### EXAMPLE 2

(rac)-[4-(7-Fluoro-3,4-dihydroisoquinolin-2(1H)-yl)piperidin-1-yl]{2-[(1-methoxybutan-2-yl)amino]pyrimidin-5-yl}methanone of the formula

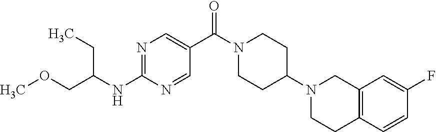 [SMILES: CCC(COC)Nc1ncc(C(=O)N2CCC(N3CCc4ccc(F)cc4C3)CC2)cn1]

### EXAMPLE 3

(rac)-[4-(6-Fluoro-3,4-dihydroisoquinolin-2(1H)-yl)piperidin-1-yl]{2-[(1-methoxybutan-2-yl)amino]pyrimidin-5-yl}methanone of the formula

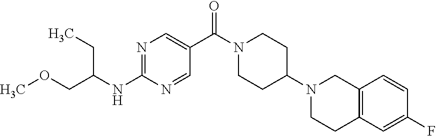 [SMILES: CCC(COC)Nc1ncc(C(=O)N2CCC(N3CCc4cc(F)ccc4C3)CC2)cn1]

### EXAMPLE 4

(rac)-{2-[(1-Methoxybutan-2-yl)amino]pyrimidin-5-yl}[4-(7-methoxy-3,4-dihydroisoquinolin-2(1H)-yl)piperidin-1-yl]methanone of the formula

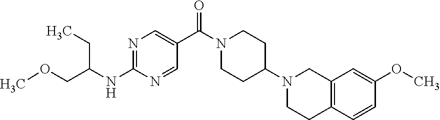

### EXAMPLE 5

(rac)-{2-[(1-Methoxybutan-2-yl)amino]pyrimidin-5-yl}[4-(6-methoxy-3,4-dihydroisoquinolin-2(1H)-yl)piperidin-1-yl]methanone of the formula

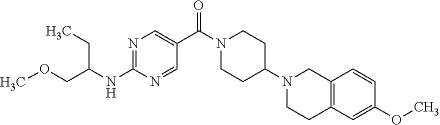

### EXAMPLE 6

(rac)-[4-(3,4-Dihydroisoquinolin-2(1H)-yl)piperidin-1-yl]{2-[(1-methoxybutan-2-yl)amino]pyrimidin-5-yl}methanone of the formula

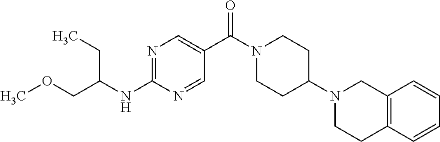 [SMILES: CCC(COC)Nc1ncc(C(=O)N2CCC(N3CCc4ccccc4C3)CC2)cn1]

### EXAMPLE 7

(rac)-[4-(3,4-Dihydroisoquinolin-2(1H)-yl)piperidin-1-yl]{2-[(1-hydroxybutan-2-yl)amino]pyrimidin-5-yl}methanone of the formula

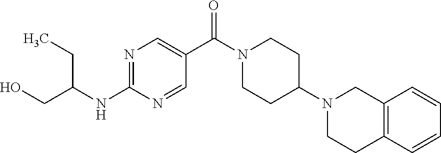 [SMILES: CCC(CO)Nc1ncc(C(=O)N2CCC(N3CCc4ccccc4C3)CC2)cn1]

### EXAMPLE 8

[4-(3,4-Dihydroisoquinolin-2(1H)-yl)piperidin-1-yl][2-(2-oxa-6-azaspiro[3.3]hept-6-yl)pyrimidin-5-yl]methanone of the formula

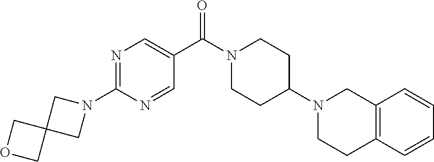 [SMILES: O=C(c1cnc(N2CC3(COC3)C2)nc1)N1CCC(N2CCc3ccccc3C2)CC1]

### EXAMPLE 9

[4-(7-Fluoro-3,4-dihydroisoquinolin-2(1H)-yl)piperidin-1-yl][2-(2-oxa-6-azaspiro[3.3]hept-6-yl)pyrimidin-5-yl]methanone of the formula

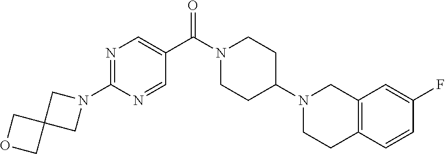 [SMILES: O=C(c1cnc(N2CC3(COC3)C2)nc1)N1CCC(N2CCc3ccc(F)cc3C2)CC1]

### EXAMPLE 10

[4-(6-Methoxy-3,4-dihydroisoquinolin-2(1H)-yl)piperidin-1-yl][2-(2-oxa-6-azaspiro[3.3]hept-6-yl)pyrimidin-5-yl]methanone of the formula

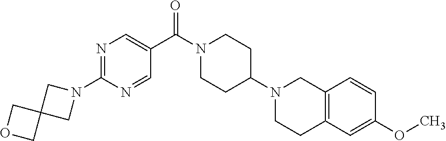 [SMILES: COc1ccc2c(c1)CCN(C1CCN(C(=O)c3cnc(N4CC5(COC5)C4)nc3)CC1)C2]

### EXAMPLE 11

1-(5-{[4-(3,4-Dihydroisoquinolin-2(1H)-yl)piperidin-1-yl]carbonyl}pyrimidin-2-yl)-D-proline hydrochloride of the formula

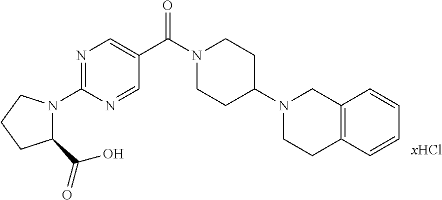 [SMILES: O=C(O)[C@H]1CCCN1c1ncc(C(=O)N2CCC(N3CCc4ccccc4C3)CC2)cn1]

### EXAMPLE 12

[4-(3,4-Dihydroisoquinolin-2(1H)-yl)piperidin-1-yl][2-(1,1-dioxidothiomorpholin-4-yl)pyrimidin-5-yl]methanone of the formula

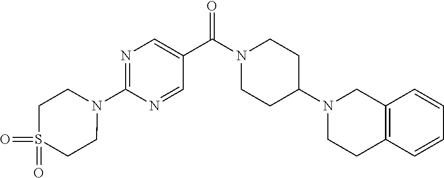 [SMILES: O=C(c1cnc(N2CCS(=O)(=O)CC2)nc1)N1CCC(N2CCc3ccccc3C2)CC1]

### EXAMPLE 13

[4-(3,4-Dihydroisoquinolin-2(1H)-yl)piperidin-1-yl][2-(2,6-dimethylmorpholin-4-yl)pyrimidin-5-yl]methanone (cis isomer) of the formula

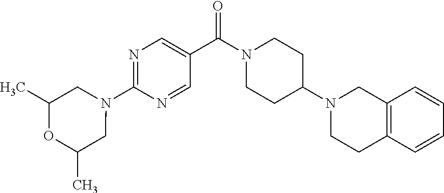 [SMILES: CC1CN(c2ncc(C(=O)N3CCC(N4CCc5ccccc5C4)CC3)cn2)CC(C)O1]

### EXAMPLE 14

[4-(3,4-Dihydroisoquinolin-2(1H)-yl)piperidin-1-yl][2-(2,6-dimethylmorpholin-4-yl)pyrimidin-5-yl]methanone (trans isomer) of the formula

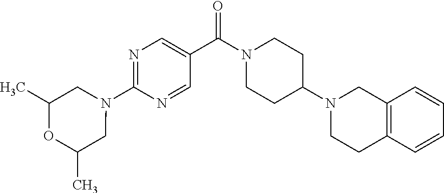 [SMILES: CC1CN(c2ncc(C(=O)N3CCC(N4CCc5ccccc5C4)CC3)cn2)CC(C)O1]

### EXAMPLE 15

[4-(3,4-Dihydroisoquinolin-2(1H)-yl)piperidin-1-yl][2-(2,2-dimethylmorpholin-4-yl)pyrimidin-5-yl]methanone of the formula

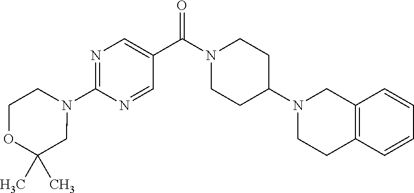 [SMILES: CC1(C)CN(c2ncc(C(=O)N3CCC(N4CCc5ccccc5C4)CC3)cn2)CCO1]

Especially preferred is Example 8 of WO2015091414, [4-(3,4-Dihydroisoquinolin-2(1H)-yl)piperidin-1-yl][2-(2-oxa-6-azaspiro[3.3]hept-6-yl)pyrimidin-5-yl]methanone of the formula

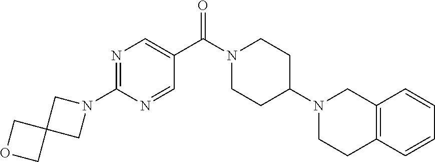 [SMILES: O=C(c1cnc(N2CC3(COC3)C2)nc1)N1CCC(N2CCc3ccccc3C2)CC1]

for use in the treatment and/or prophylaxis of peripheral artery diseases (PAD) and/or critical limb ischemia (CLI).

A further aspect of the present invention are compounds according to formula (I) for the use in patients suffering from peripheral artery diseases (PAD) and/or critical limb ischemia (CLI).

A further aspect of the present invention are pharmaceutical formulations comprising at least one compound of formula (I) for the use in patients suffering from peripheral artery diseases (PAD) and/or critical limb ischemia (CLI).

A further aspect of the present invention is the use of compounds according to formula (I) for the manufacture of a medicament for the treatment and/or prophylaxis of peripheral artery diseases (PAD) and/or critical limb ischemia (CLI).

The present invention is also directed to combinations comprising adrenoreceptor α_{2C} receptor antagonists of the formula (I) and one or more chymase inhibitors known from WO2013167495 wherein at least one chymase inhibitor is selected from the group consisting of:

1-(1,3-dimethyl-2-oxo-2,3-dihydro-1H-benzimidazol-5-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of the formula (A)

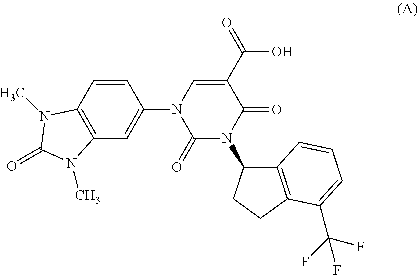 [SMILES: Cn1c(=O)n(C)c2cc(-n3cc(C(=O)O)c(=O)n([C@@H]4CCc5c4cccc5C(F)(F)F)c3=O)ccc21]

1-(6-fluoro-1,3-dimethyl-2-oxo-2,3-dihydro-1H-benzimidazol-5-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of the formula (B)

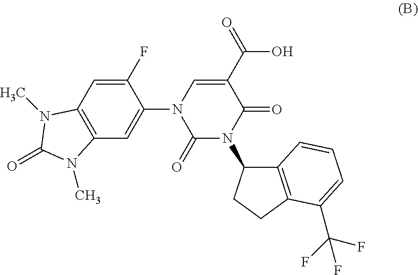 [SMILES: Cn1c(=O)n(C)c2cc(-n3cc(C(=O)O)c(=O)n([C@@H]4CCc5c4cccc5C(F)(F)F)c3=O)c(F)cc21]

1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of the formula (C)

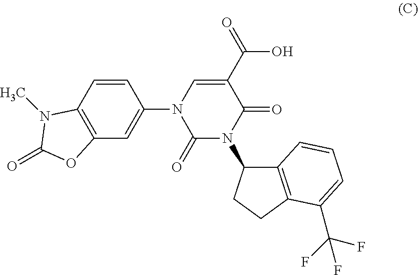 [SMILES: Cn1c(=O)oc2cc(-n3cc(C(=O)O)c(=O)n([C@@H]4CCc5c4cccc5C(F)(F)F)c3=O)ccc21]

2,4-dioxo-3-[(1R)-4-(trifluoromethyl)-2,3-dihydro-1H-inden-1-yl]-1-(1,3,3-trimethyl-2-oxo-2,3-dihydro-1H-indol-5-yl)-1,2,3,4-tetrahydropyrimidine-5-carboxylic acid (R enantiomer) of the formula (D)

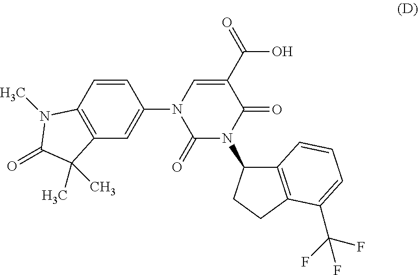 [SMILES: CN1C(=O)C(C)(C)c2cc(-n3cc(C(=O)O)c(=O)n([C@@H]4CCc5c4cccc5C(F)(F)F)c3=O)ccc21]

1-(1′-methyl-2′-oxo-1′,2′-dihydrospiro[cyclopropane-1,3′-indole]-5′-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of the formula (E)

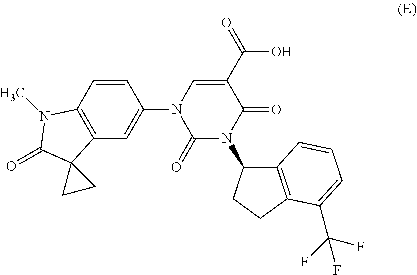 [SMILES: CN1C(=O)C2(CC2)c2cc(-n3cc(C(=O)O)c(=O)n([C@@H]4CCc5c4cccc5C(F)(F)F)c3=O)ccc21]

1-(3-methyl-2-oxo-2,3-dihydro-1,3-benzothiazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of the formula (F)

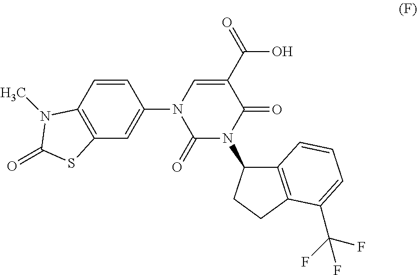 [SMILES: Cn1c(=O)sc2cc(-n3cc(C(=O)O)c(=O)n([C@@H]4CCc5c4cccc5C(F)(F)F)c3=O)ccc21]

and

ethyl 1-(3-methyl-2-oxo-2,3-dihydro-1,3-benzoxazol-6-yl)-2,4-dioxo-3-[(1R)-4-(trifluoro-methyl)-2,3-di-hydro-1H-inden-1-yl]-1,2,3,4-tetrahydropyrimidine-5-carboxylate (R enantiomer) of the formula (G)

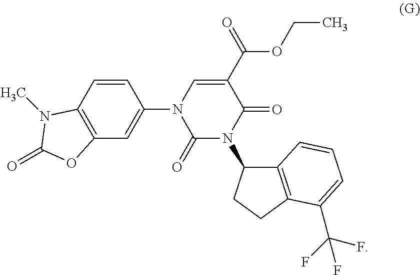 [SMILES: CCOC(=O)c1cn(-c2ccc3c(c2)oc(=O)n3C)c(=O)n([C@@H]2CCc3c2cccc3C(F)(F)F)c1=O]

Preferred combinations (adrenoreceptor α_{2C} receptor antagonists and chymase inhibitor) are: Example 1 and (A), Example 1 and (B), Example 1 and (C), Example 1 and (D), Example 1 and (E), Example 1 and (F), Example 1 and (G), Example 2 and (A), Example 2 and (B), Example 2 and (C), Example 2 and (D), Example 2 and (E), Example 2 and (F), Example 2 and (G), Example 3 and (A), Example 3 and (B), Example 3 and (C), Example 3 and (D), Example 3 and (E), Example 3 and (F), Example 3 and (G), Example 4 and (A), Example 4 and (B), Example 4 and (C), Example 4 and (D), Example 4 and (E), Example 4 and (F), Example 4 and (G), Example 5 and (A), Example 5 and (B), Example 5 and (C), Example 5 and (D), Example 5 and (E), Example 5 and (F), Example 5 and (G), Example 6 and (A), Example 6 and (B), Example 6 and (C), Example 6 and (D), Example 6 and (E), Example 6 and (F), Example 6 and (G), Example 7 and (A), Example 7 and (B), Example 7 and (C), Example 7 and (D), Example 7 and (E), Example 7 and (F), Example 7 and (G), Example 8 and (A), Example 8 and (B), Example 8 and (C), Example 8 and (D), Example 8 and (E), Example 8 and (F), Example 8 and (G), Example 9 and (A), Example 9 and (B), Example 9 and (C), Example 9 and (D), Example 9 and (E), Example 9 and (F), Example 9 and (G), Example 10 and (A), Example 10 and (B), Example 10 and (C), Example 10 and (D), Example 10 and (E), Example 10 and (F), Example 10 and (G), Example 11 and (A), Example 11 and (B), Example 11 and (C), Example 11 and (D), Example 11 and (E), Example 11 and (F), Example 11 and (G), Example 12 and (A), Example 12 and (B), Example 12 and (C), Example 12 and (D), Example 12 and (E), Example 12 and (F), Example 12 and (G), Example 13 and (A), Example 13 and (B), Example 13 and (C), Example 13 and (D), Example 13 and (E), Example 13 and (F), Example 13 and (G), Example 14 and (A), Example 14 and (B), Example 14 and (C), Example 14 and (D), Example 14 and (E), Example 14 and (F), Example 14 and (G), Example 15 and (A), Example 15 and (B), Example 15 and (C), Example 15 and (D), Example 15 and (E), Example 15 and (F), Example 15 and (G).

More preferred combinations are: Example 1 and (C), Example 2 and (C), Example 3 and (C), Example 4 and (C), Example 5 and (C), Example 6 and (C), Example 7 and (C), Example 8 and (A), Example 8 and (B), Example 8 and (C), Example 8 and (D), Example 8 and (E), Example 8 and (F), Example 8 and (G), Example 9 and (C), Example 10 and (C), Example 11 and (C), Example 12 and (C), Example 13 and (C), Example 14 and (C), Example 15 and (C).

Especially preferred combinations are: Example 8 and (A), Example 8 and (B), Example 8 and (C), Example 8 and (D), Example 8 and (E), Example 8 and (F), Example 8 and (G).

Most preferred is the combination of Example 8 ([4-(3,4-Dihydroisoquinolin-2(1H)-yl)piperidin-1-yl][2-(2-oxa-6-azaspiro[3.3]hept-6-yl)pyrimidin-5-yl]methanone) and (C) (1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer)).

A further embodiment of the invention are combinations comprising adrenoreceptor α_{2C} receptor antagonists of the formula (I) and one or more chymase inhibitors mentioned above for use in the treatment and/or prophylaxis of peripheral artery diseases (PAD) and/or critical limb ischemia (CLI).

A further embodiment of the invention are combinations comprising adrenoreceptor α_{2C} receptor antagonists of the formula (I) and one or more chymase inhibitors mentioned above for use in the treatment and/or prophylaxis of critical limb ischemia (CLI).

Preferred is the combination of adrenoreceptor α_{2C} receptor antagonists compounds of the formula (I) with 1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of the formula (C)

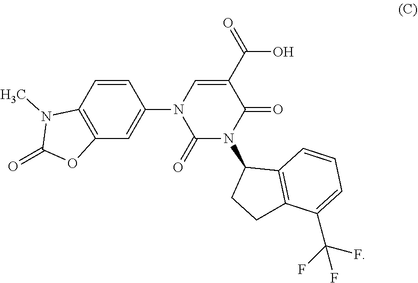 [SMILES: Cn1c(=O)oc2cc(-n3cc(C(=O)O)c(=O)n([C@@H]4CCc5c4cccc5C(F)(F)F)c3=O)ccc21]

for the use in the treatment and/or prophylaxis of critical limb ischemia (CLI).

Especially preferred is the combination of [4-(3,4-Dihydroisoquinolin-2(1H)-yl)piperidin-1-yl][2-(2-oxa-6-azaspiro[3.3]hept-6-yl)pyrimidin-5-yl]methanone and 1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) for use in the treatment and/or prophylaxis of critical limb ischemia (CLI).

Another aspect of the present invention is the combination according to the invention mentioned above for the use in patients suffering from peripheral artery diseases (PAD) and/or critical limb ischemia (CLI).

Another aspect of the present invention is the pharmaceutical formulation comprising at least one combination according to the invention mentioned above for the use in patients suffering from peripheral artery diseases (PAD) and/or critical limb ischemia (CLI).

Another aspect of the present invention are selective adrenoreceptor α_{2C} receptor antagonists alone, or in combination with chymase inhibitors for the use in patients suffering from peripheral artery diseases (PAD) and/or critical limb ischemia (CLI).

The present invention further provides a method for the treatment and/or prophylaxis of peripheral artery diseases (PAD) and/or critical limb ischemia (CLI) by administration of an effective amount of at least one adrenoreceptor α_{2C} receptor antagonist compound of the formula (I) or of a medicament comprising at least one adrenoreceptor α_{2C} receptor antagonist compound of the formula (I).

The present invention further provides a method for the treatment and/or prophylaxis of peripheral artery diseases (PAD) and/or critical limb ischemia (CLI) by administration of an effective amount of a combination comprising at least one adrenoreceptor α_{2C} receptor antagonist compound of the formula (I) and one or more chymase inhibitors or of a medicament comprising a combination comprising at least one adrenoreceptor α_{2C} receptor antagonists compound of formula (I) and one or more chymase inhibitors.

Another preferred embodiment of the invention is a kit comprising at least one adrenoreceptor α_{2C} receptor antagonist as indicated above or a combination as indicated above for the use in the treatment and/or prophylaxis of peripheral artery diseases (PAD) and/or critical limb ischemia (CLI).

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in suitable administration forms for these administration routes.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Suitable administration forms for parenteral administration include injection and infusion formulations in the form of solutions, suspensions, emulsions, lyophilizates or sterile powders.

Oral administration is preferred.

In the exemplary use of the compounds of the formula (I) for promoting diabetic wound healing, in particular for promoting wound healing of diabetic foot ulcers, preference, in addition to oral administration, is also given to administration in the form of a topical formulation.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In general, it has been found to be advantageous in the case of oral administration to administer amounts of from about 0.1 to 250 mg per 24 hours, preferably 0.1 to 50 mg per 24 hours, to achieve effective results. The dose may be divided into a plurality of administrations per day. Examples are administrations twice or three times per day.

It may nevertheless be necessary where appropriate to deviate from the stated amounts, specifically as a function of the body weight, route of administration, individual response to the active compound, nature of the preparation and time or interval over which administration takes place.

### A) EXAMPLES

### Abbreviations

ApoE apolipoprotein E knockout

B.I.D “bis in die”

ca. circa

CLI critical limb ischemia

Ctrl. Control

Ex. Example

eq. equivalent(s)

h hour(s)

min minute(s)

PEG polyethyleneglycol

POD post-wounding days

PU Perfusion Unit

SEM standard error of mean

STZ streptozotocin

ZDF Zucker diabetic fatty

### A) Compounds

### Compound of Example 8: According to WO 2015/091414:

[4-(3,4-Dihydroisoquinolin-2(1H)-yl)piperidin-1-yl][2-(2-oxa-6-azaspiro[3.3]hept-6-yl)pyrimidin-5-yl]methanone

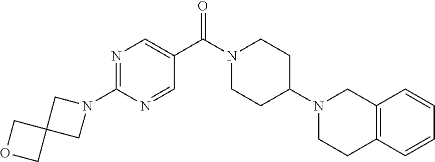 [SMILES: O=C(c1cnc(N2CC3(COC3)C2)nc1)N1CCC(N2CCc3ccccc3C2)CC1]

Pluronic® F 127 30% gel: WFI 70%

### B) Assessment of Physiological Efficacy

CLI patients are suffering from poorly healing wounds finally leading to gangrene. To address these symptoms, animal studies were performed in ischemic wounds in rabbits as well as in surgically induced wounds in diabetic pigs to evaluate the effects of our drug on wounds with different etiologies. The diabetic pig model is a very severe model which is considered to be the most predictive model for human wounds.

The suitability of the compounds according to the invention for treating PAD/CLI can be demonstrated in the following assay systems:

### B-1) In vivo Assays

### B-1a) Wound Healing in Streptozotocin (STZ)-Challenged Minipigs

Wounds (twelve 15.15-mm full-thickness wounds) were induced in STZ-challenged minipigs. STZ challenge (160 mg/kg) was performed 14 days before wounding in 15 animals, two out of 15 animals had to be sacrificed due to blood glucose instability. The remaining 13 animals (body weight around 25 kg: 27.92±2.35 kg on the day of STZ dosing, Day 0: 24.35±2.14 kg and Day 17: 23.27±2.24 kg) were randomized to 3 groups:

Group I) placebo: Animal 1, 8, 10 and 13; Group II) 0.6 mg/kg Compound of Ex 8: Animal 2, 7, 11 and 12; Group III) 0.1 mg/kg Compound of Ex. 8: Animal 3, 4, 6, 14 and 15

Oral gavage was done by gelatin capsules containing Compound of Ex. 8 or lactose as placebo (25 kg body weight was assumed for all animals).

Topical treatment was performed using Compound of Ex. 8 in a concentration of 0.06 mg/mL in a Pluronic® gel matrix or as placebo Pluronic® gel at a volume of 1 mL per wound. The whole study was performed in a blinded manner. The 12 wounds per animal were treated according to Table 1 to combine oral treatment with and without topical treatment in this study. Wounds were closed by a sterile reclosable bag system. Animals were treated orally BID for 17 days starting after wounding (Day 0) and topically once daily on Days 0, 2, 5, 8, 11 and 14. On Day 17, wounds were harvested and animals sacrificed. Blood glucose levels during the study were in the range between 200 and 450 mg/dL and did not differ between the three groups.

TABLE 1
Scheme of wounding in all three animal groups

| | |
|---|---|
| wounds 1 to 6 | without topical treatment |
| wounds 7, 9 and 11 | topically treated with Pluronic ® placebo gel |
| wounds 8, 10 and 12 | with Pluronic ® gel containing Compound |
| | of Ex. 8 (0.06 mg/mL) |

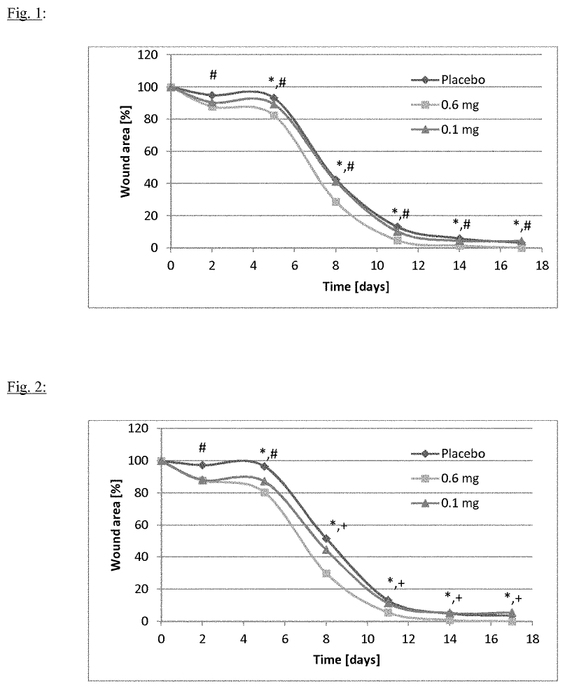

The primary endpoint of this study was wound size over study time. Wound size was evaluated via photographs and evaluated by photoshop under blinded conditions. Wound size was shown in absolute numbers as well as percent size vs. wound size on Day 0. In addition, re-epithelialization as well as wound contracture were determined The mean of all 12 wounds per animal was compared to Day 0. All animals showed similar wound healing courses until Day 5. Between Day 5 and Day 11, a differentiation between the animals was observed. The Compound of Ex. 8 0.6 mg/kg group showed a quicker wound healing compared to the 2 other groups. The 0.1 mg/kg group showed a greater heterogeneity. On Day 17, two animals of the 0.6 mg/kg group were characterized by a complete and stable re-epithelialization, whereas all other animals still showed at least some wounds, which were only partially closed. The mean of all wounds between the 3 groups showed a significantly improved wound healing between the Compound of Ex. 8 0.6 mg/kg group and placebo for all days (FIG. 1).

The comparison of the wounds (Wounds 1 to 6), which were not treated topically, revealed a better differentiation between the groups, showing the effect of oral treatment alone. All values of the 0.6 mg/kg Compound of Ex. 8 group were significantly better compared to the placebo group. On Day 8, the difference was about 21.6%. Moreover, the Compound of Ex. 8 0.1 mg/kg group showed a significant improvement vs. placebo on Day 2 (P=0.011) and Day 5 (P=0.014). On Days 8, 11, 14 and 17 the Compound of Ex. 8 0.6 mg/kg group was significantly better compared to the 0.1 mg/kg group, showing a dose-dependent effect (FIG. 2).

Further analysis of wounds revealed a therapeutic effect of the Pluronic® gel. However, the main effect in the study was achieved by oral treatment.

The improved wound healing by Compound of Ex. 8 was partially mediated by an improvement in wound contracture being significant for the Compound of Ex. 8 0.6 mg/kg group on Day 2 (P=0.003); Day 5 (P=0.005); Day 8 (P=0.033), and Day 11 (P=0.012) and for the Compound of Ex. 8 0.1 mg/kg group in addition on Day 17 (P=0.014) [Day 2 (P=0.002); Day 5 (P=0.0013); Day 8 (P=0.046), and Day 11 (P=0.005)]. Histological evaluation of the study revealed more advanced wound healing for the Compound of Ex. 8 (0.6 mg) treated wounds compared to placebo. This was determined by (i) reduced cell proliferation, (ii) higher re-epithelialization level, (iii) increased differentiation level of the epidermis, (iv) reduced inflammation, (v) lower collagen III/I ratio, and (vi) more detectable vessels and (vii) increased structuring of basement membranes defined by laminin detection.

### B-1b) Wound Healing in the Ischemic Rabbit Ear Model

Ischemic wounds (three 7 mm full-thickness dermal punches on the inner surface of both ears) were induced in New Zealand white rabbits. Oral gavage was done through a nasogastric tube or catheter. 20 rabbits were treated with Compound of Ex. 8 (0.3 mg/kg; n=10) or vehicle (10% ethanol, 30% PEG 400, 60% tap water, n=10) through the catheter daily at multiple time points [post-wounding days (POD) 3, 4, 5, 6 and 7]. Wounds were harvested on POD 10. For histologic analysis, one-half of wounds including the surrounding normal skin in rectangular shape was taken. One-half of the wound was frozen for further molecular analysis. Tissues underwent routine processing, paraffin embedding, and sectioning. A 4 μm cross section through the center of each rectangular biopsy was taken. The tissues were stained with hematoxylin and eosin and examined under light microscopy. Several histomorphometric measurements were determined, using a digital image analysis system (NIS-Elements Basic Research; Nikon Instech Co, Kanagawa, Japan) at 2 fold and 10 fold magnification. Each parameter was measured in a blinded manner.

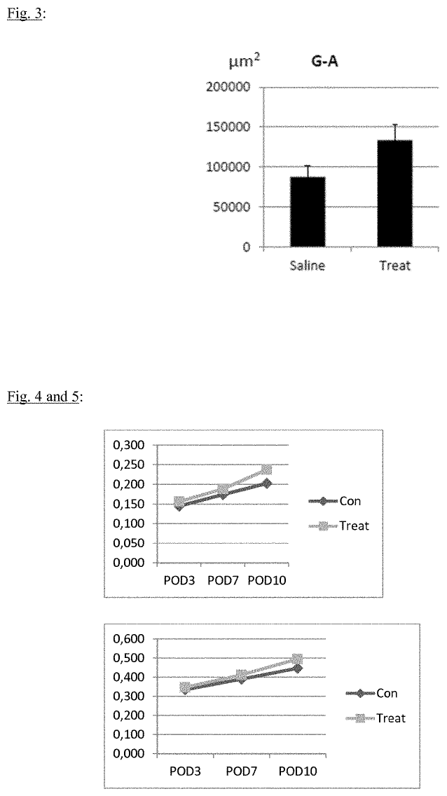

Regarding histological data, 9 vehicle-alone animals and 10 animals treated with Compound of Ex. 8 were compared. Regarding epithelial gap, no significant difference was observed between the Compound of Ex. 8-treated (0.3 mg/kg) group (5,212+213 μm) and the control group (5,348+191 μm). Regarding the granulation area, the Compound of Ex. 8-treated (0.3 mg/kg) group showed a larger granulation area (133,630+20,208 μm²) compared to the control group (87,582+14,419 μm²) (FIG. 3). However, the difference did not reach statistical significance (P=0.07).

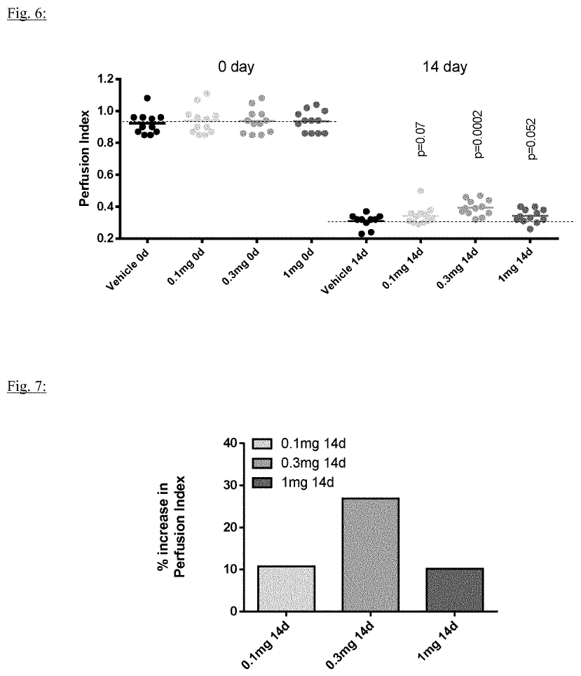

In addition, tissue oxygen tension and blood flow were examined by the OxyLite® system (Oxford Optronix). Oxygen tension and blood flow were measured on POD 3, 7 and 10 in each rabbit. The oxygen tension unit is mmHg and the blood flow unit is blood PUs. The results were presented as ratio of O2-ischemia/O2-non-ischemia, or flow-ischemia/flow-non-ischemia. Analysis was performed by comparison between control and treatment groups on POD 3, 7 and 10. Regarding oxygen tension, the Compound of Ex. 8-treated (0.3 mg/kg) group showed a higher oxygen tension ratio (0.238) compared to the control group (0.211) on POD 10. However, this difference did not reach statistical significance (P=0.079). Regarding blood flow, the Compound of Ex. 8-treated (0.3 mg/kg) group showed a significantly higher blood flow ratio (0.495) compared to the control group (0.447) on POD 10 (P=0.018) (FIGS. 4 and 5).

A further analysis was performed by comparing the change from POD 3 to POD 10 between the control and treatment groups in the same rabbit. Regarding oxygen tension, the Compound of Ex. 8-treated (0.3 mg/kg) group showed an increased oxygen tension (0.082) compared to the control group (0.066). However, the difference did not reach statistical significance (P=0.322). Regarding blood flow, the Compound of Ex. 8-treated (0.3 mg/kg) group showed an increased blood flow (0.149) compared to the control group (0.113). However, the difference did not reach statistical significance (P=0.108).

### B-1c) Effects on Ankle-Brachial Index in ZDF Rats

In a unilateral hindlimb ischemia model, Compound of Ex. 8 (0.1, 0.3, and 1 mg/kg) induced an increase in perfusion indices at all doses (FIG. 6). The 0.3 mg/kg dose was most effective leading to a significant increase in perfusion pressure (FIG. 6). Compound of Ex. 8 induced a 10 to 30% improvement in the perfusion index (FIG. 7). Regarding muscle function, Compound of Ex. 8 showed a slight tendency toward improved muscle function in the ligated limb. Corresponding plasma concentrations were measured between 4 and 47 μg/L.

### B-1d) Effects on Running Distance in Apolipoprotein Knockout Mice

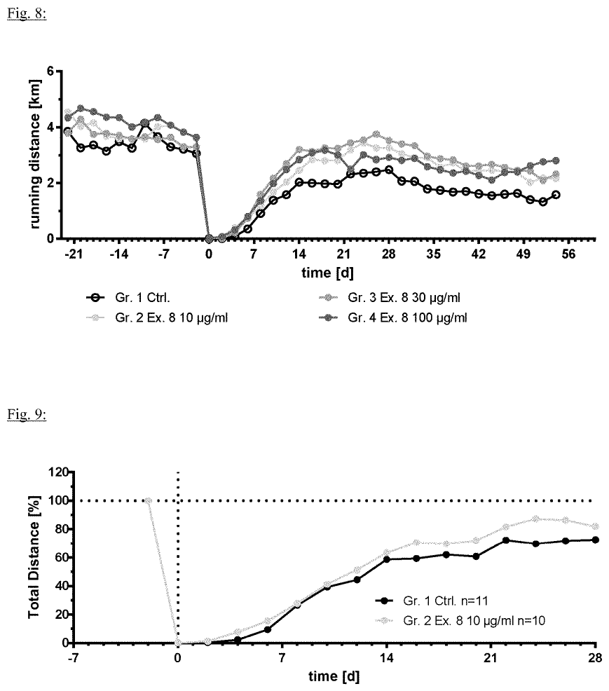

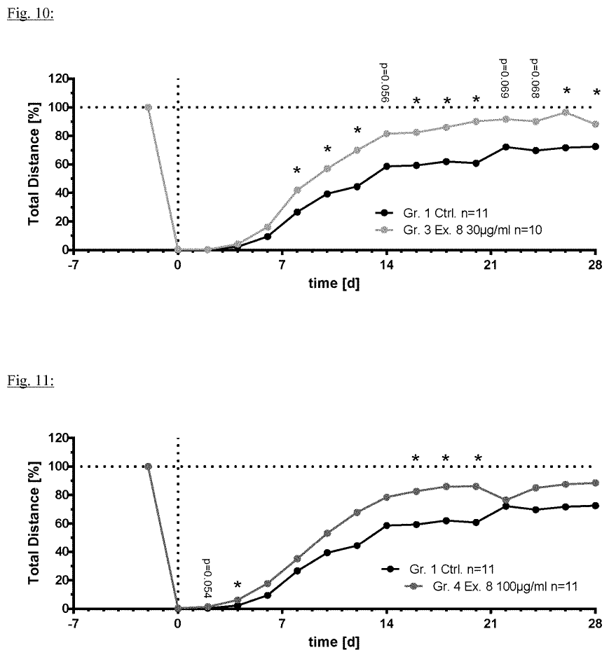

Compound of Ex. 8 after oral administration of 10, 30 and 100 μg/mL induced an increase in running distance in atherosclerotic ApoE-/- (apolipoprotein E knockout) mice after induction of hindlimb ischemia (FIG. 8). To evaluate effects more closely, the percent change in running distance compared to the run-in values (2 week run-in period before ligature) of all animals were analyzed. Compound of Ex. 8 at 10 μg/ml showed a tendency toward an improvement in the total running distance (FIGS. 9). 30 and 100 μg/kg Compound of Ex. 8 induced a significant improvement in running distance (FIG. 10 and FIG. 11) on several days during the 4 week observation period. The area under the curve from Day 0 to Day 28 for the 30 and 100 μg/kg Compound of Ex. 8 groups showed significant improvements compared to placebo.

### EXPLANATION OF THE FIGURES

FIG. 1: Change of wound size over time shown as mean±SEM of all wounds of the 3 different groups (12 wounds per animal, 4 to 5 animals per group) Day 0 to Day 28 for the 30 and 100 μg/kg
*P<0.05 for 0.6 mg Compound of Ex. 8 versus 0.1 mg, # P<0.05 for 0.6 mg Compound of Ex. 8 versus placebo; SEM=standard error of mean

FIG. 2: Change of wound size over time shown as mean±SEM of all wounds without topical treatment (Wounds 1 to 6) of the 3 different groups (6 wounds per animal, 4 to 5 animals per group)
*P<0.05 for 0.6 mg Compound of Ex. 8 versus placebo, # P<0.05 for 0.1 mg Compound of Ex. 8 versus placebo, +P<0.05 for 0.6 mg Compound of Ex. 8 versus 0.1 mg Compound of Ex. 8; SEM=standard error of mean

FIG. 3: Effects of Compound of Ex. 8 on granulation area (G-A) after wound induction in the ischemic rabbit ear model (mean+SEM; 9/10 animals and 53/56 wounds per group)
Saline=vehicle, Treat=Compound of Ex. 8, 0.3 mg/kg

FIG. 4/5: Effects of Compound of Ex. 8 on oxygen tension and blood flow after wound induction in the ischemic rabbit ear model (mean+SEM; 10 animals and 20 examination sites per group)
Con=control (saline=vehicle), Treat=Compound of Ex. 8, 0.3 mg/kg

FIG. 6: Effects of Compound of Ex. 8 on perfusion pressure indices. Shown are baseline value (Od) as well as perfusion pressure indices after ligature of the left iliac artery and 2 weeks of daily treatment (14 d) with Compound of Ex. 8 in diabetic ZDF fa/fa rats (raw data; 10 animals)
Student t-test, Compound of Ex. 8 versus vehicle; ZDF=Zucker diabetic fatty

FIG. 7: Percent change in perfusion pressure indices by Compound of Ex. 8 after 14 days (14 d) of daily treatment compared to baseline values (mean±SEM; 10 animals)
SEM=standard error of mean; ZDF=Zucker diabetic fatty

FIG. 8: Effects of Compound of Ex. 8 on daily running distance in atherosclerotic ApoE-/- mice before and after induction of bilateral hindlimb ischemia (mean±SEM; 10 to 11 animals)
ApoE-/-=apolipoprotein E knockout; Ctrl.=control; SEM=standard error of mean

FIG. 9: Effects of Compound of Ex. 8 (10 μg/mL) on daily running distance in atherosclerotic ApoE-/- mice before and after induction of bilateral hindlimb ischemia (mean±SEM; 10 to 11 animals)
*P<0.05 (Student t-test, Compound of Ex. 8 versus Ctrl.); ApoE-/-=apolipoprotein E knockout; Ctrl.=control; SEM=standard error of mean

FIG. 10: Effects of Compound of Ex. 8 (30 μg/mL) on daily running distance in atherosclerotic ApoE-/- mice before and after induction of bilateral hindlimb ischemia (mean±SEM; 10 to 11 animals)
*P<0.05 (Student t-test, Compound of Ex. 8versus Ctrl.); ApoE-/-=apolipoprotein E knockout; Ctrl.=control; SEM=standard error of mean

FIG. 11: Effects of Compound of Ex. 8 (100 μg/mL) on daily running distance in atherosclerotic ApoE-/- mice before and after induction of bilateral hindlimb ischemia (mean±SEM; 11 animals)
*P<0.05 (Student t-test, Compound of Ex. 8 versus Ctrl.); ApoE-/-=apolipoprotein E

1. A method for treatment and/or prophylaxis of peripheral artery diseases (PAD) and/or critical limb ischemia (CLI) comprising administering to a patient in need thereof an effective amount of at least one selective adrenoreceptor α_{2C} receptor antagonist of formula (I) 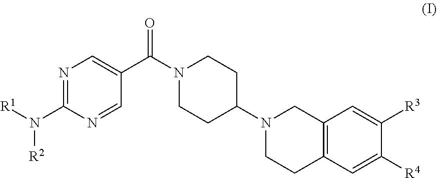
wherein
R¹ is C₁-C₆-alkyl or C₃-C₅-cycloalkyl,
where alkyl is substituted by 1 to 2 substituents independently of one another selected from the group consisting of hydroxy and C₁-C₄-alkoxy
and
R² is hydrogen or C₁-C₄-alkyl,
or
R¹ and R² together with the nitrogen atom to which they are attached form a 4- to 7-membered N-heterocycle,
where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy, and halogen,
or
where the N-heterocycle may have two substituents which, together with the carbon atom of the N-heterocycle to which they are jointly attached, form a 4- to 6-membered heterocycle,
where this heterocycle for its part may be substituted by 1 to 3 substituents independently of one another selected from the group consisting of oxo, methyl, and ethyl,
R³ is hydrogen, fluorine, methoxy, or ethoxy,
and
R⁴ is hydrogen, fluorine, methoxy, or ethoxy,
or a salt thereof, a solvate thereof, or a solvate of the salt thereof.

2. The method according to claim 1, wherein
R¹ is C₂-C₄-alkyl,
where alkyl is substituted by a substituent selected from the group consisting of hydroxy and methoxy,
and
R² is hydrogen,
or
R¹ and R² together with the nitrogen atom to which they are attached form an azetidine, pyrrolidine, morpholine, or 1,1-dioxidothiomorpholine,
where azetidine, pyrrolidine, morpholine, or 1,1-dioxidothiomorpholine may be substituted by 1 to 2 substituents selected independently from the group consisting of hydroxycarbonyl, methyl, trifluoromethyl, methoxy, and methoxymethyl,
or
R¹ and R² together with the nitrogen atom to which they are attached form an azetidine,
where the azetidine may have two substituents which, together with the carbon atom of the azetidine to which they are jointly attached, form an oxetane or 1,1-dioxidothietane,
R³ is hydrogen, fluorine, or methoxy,
and
R⁴ is hydrogen,
or
R³ is hydrogen,
and
R⁴ is hydrogen, fluorine, or methoxy.

3. The method according to claim 1, wherein
R¹ is C₂-C₆-alkyl,
where alkyl is substituted by a substituent selected from the group consisting of hydroxy, methoxy, and ethoxy,
and
R² is hydrogen,
or
R¹ and R² together with the nitrogen atom to which they are attached form an azetidine, pyrrolidine, piperidine, azepane, piperazine, morpholine, thiomorpholine, 1-oxidothiomorpholine, or 1,1-dioxidothiomorpholine,
where azetidine, pyrrolidine, piperidine, azepane, piperazine, morpholine, thiomorpholine, 1-oxidothiomorpholine, and 1,1-dioxidothiomorpholine may be substituted by 1 to 2 substituents independently of one another selected from the group consisting of hydroxy, hydroxycarbonyl, C₁-C₃-alkyl, and methoxy,
or
where azetidine, pyrrolidine, piperidine, azepane, piperazine, and morpholine may have two substituents which, together with the carbon atom of the azetidine, pyrrolidine, piperidine, azepane, piperazine, or morpholine to which they are jointly attached, form an azetidine or oxetane,
where this azetidine or oxetane for its part may be substituted by 1 to 2 substituents independently of one another selected from the group consisting of methyl and ethyl,
R³ is hydrogen,
and
R⁴ is hydrogen, fluorine, or methoxy,
or
R³ is hydrogen, fluorine, or methoxy,
and
R⁴ is hydrogen.

4. A method for treatment and/or prophylaxis of peripheral artery diseases (PAD) and/or critical limb ischemia (CLI) comprising administering to a patient in need thereof an effective amount of [4-(3,4-Dihydroisoquinolin-2(1H)-yl)piperidin-1-yl][2-(2-oxa-6-azaspiro[3.3]hept-6-yl)pyrimidin-5-yl]methanone of formula

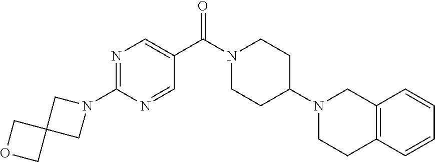 [SMILES: O=C(c1cnc(N2CC3(COC3)C2)nc1)N1CCC(N2CCc3ccccc3C2)CC1]

5. (canceled)

6. A method for treatment and/or prophylaxis of peripheral artery diseases (PAD) and/or critical limb ischemia (CLI) comprising administering to a patient in need thereof an effective amount of a pharmaceutical formulation comprising at least one selective adrenoreceptor α_{2C} receptor antagonist of formula (I) 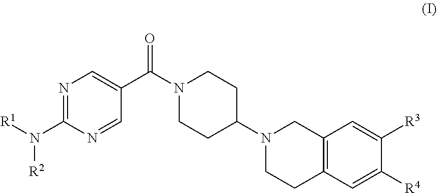
wherein
R¹ is C₁-C₆-alkyl or C₃-C₅-cycloalkyl,
where alkyl is substituted by 1 to 2 substituents independently of one another selected from the group consisting of hydroxy and C₁-C₄-alkoxy
and
R² is hydrogen or C₁-C₄-alkyl,
or
R¹ and R² together with the nitrogen atom to which they are attached form a 4- to 7-membered N-heterocycle,
where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy, and halogen, or
where the N-heterocycle may have two substituents which, together with the carbon atom of the N-heterocycle to which they are jointly attached, form a 4- to 6-membered heterocycle,
where this heterocycle for its part may be substituted by 1 to 3 substituents independently of one another selected from the group consisting of oxo, methyl, and ethyl,
R³ is hydrogen, fluorine, methoxy, or ethoxy,
and
R⁴ is hydrogen, fluorine, methoxy, or ethoxy,
or a salt thereof, a solvate thereof, or a solvate of the salt thereof.

7. A combination comprising an adrenoreceptor α_{2C} receptor antagonist of formula (I) and one or more chymase inhibitor, 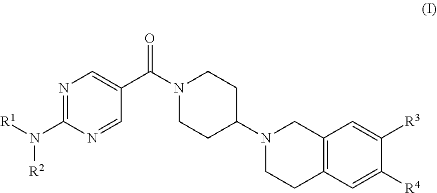
wherein
R¹ is C₁-C₆-alkyl or C₃-C₅-cycloalkyl,
where alkyl is substituted by 1 to 2 substituents independently of one another selected from the group consisting of hydroxy and C₁-C₄-alkoxy
and
R² is hydrogen or C₁-C₄-alkyl,
or
R¹ and R² together with the nitrogen atom to which they are attached form a 4- to 7-membered N-heterocycle,
where the N-heterocycle may be substituted by 1 to 3 substituents independently of one another selected from the group consisting of oxo, hydroxy, monofluoromethyl, difluoromethyl, trifluoromethyl, hydroxycarbonyl, tert-butoxycarbonyl, aminocarbonyl, C₁-C₄-alkyl, C₁-C₄-alkoxy, and halogen,
or
where the N-heterocycle may have two substituents which, together with the carbon atom of the N-heterocycle to which they are jointly attached, form a 4- to 6-membered heterocycle,
where this heterocycle for its part may be substituted by 1 to 3 substituents independently of one another selected from the group consisting of oxo, methyl, and ethyl,
R³ is hydrogen, fluorine, methoxy, or ethoxy,
and
R⁴ is hydrogen, fluorine, methoxy, or ethoxy,
or a salt thereof, a solvate thereof, or a solvate of the salt thereof, and
wherein at least one chymase inhibitor is selected from the group consisting of:
1-(1,3-dimethyl-2-oxo-2,3-dihydro-1H-benzimidazol-5-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (A) 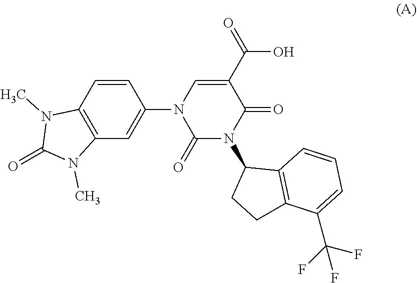 [SMILES: Cn1c(=O)n(C)c2cc(-n3cc(C(=O)O)c(=O)n([C@@H]4CCc5c4cccc5C(F)(F)F)c3=O)ccc21]
1-(6-fluoro-1,3-dimethyl-2-oxo-2,3-dihydro-1H-benzimidazol-5-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (B) 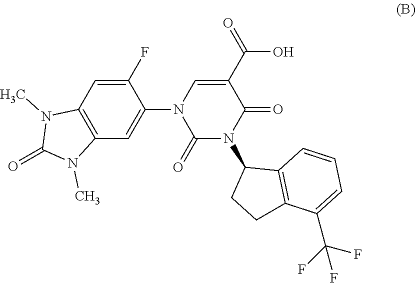 [SMILES: Cn1c(=O)n(C)c2cc(-n3cc(C(=O)O)c(=O)n([C@@H]4CCc5c4cccc5C(F)(F)F)c3=O)c(F)cc21]
1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (C) 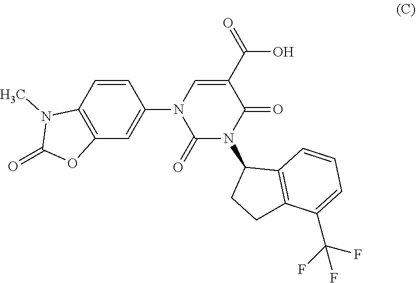 [SMILES: Cn1c(=O)oc2cc(-n3cc(C(=O)O)c(=O)n([C@@H]4CCc5c4cccc5C(F)(F)F)c3=O)ccc21]
2,4-dioxo-3-[(1R)-4-(trifluoromethyl)-2,3-dihydro-1H-inden-1-yl]-1-(1,3,3-trimethyl-2-oxo-2,3-dihydro-1H-indol-5-yl)-1,2,3,4-tetrahydropyrimidine-5-carboxylic acid (R enantiomer) of formula (D) 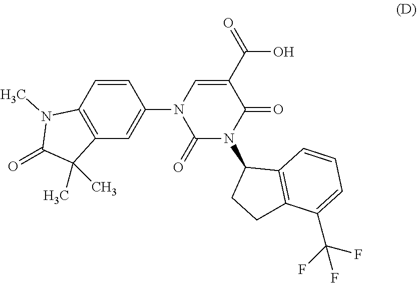 [SMILES: CN1C(=O)C(C)(C)c2cc(-n3cc(C(=O)O)c(=O)n([C@@H]4CCc5c4cccc5C(F)(F)F)c3=O)ccc21]
1-(1′-methyl-2′-oxo-1′,2′-dihydrospiro[cyclopropane-1,3′-indole]-5′-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (E) 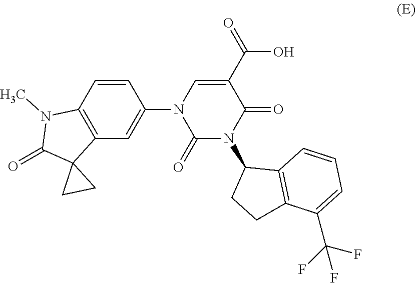 [SMILES: CN1C(=O)C2(CC2)c2cc(-n3cc(C(=O)O)c(=O)n([C@@H]4CCc5c4cccc5C(F)(F)F)c3=O)ccc21]
1-(3-methyl-2-oxo-2,3-dihydro-1,3-benzothiazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (F) 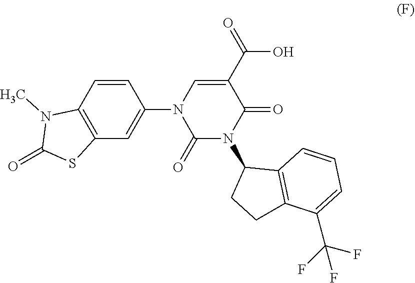 [SMILES: Cn1c(=O)sc2cc(-n3cc(C(=O)O)c(=O)n([C@@H]4CCc5c4cccc5C(F)(F)F)c3=O)ccc21]
and
ethyl 1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylate (R enantiomer) of formula (G)

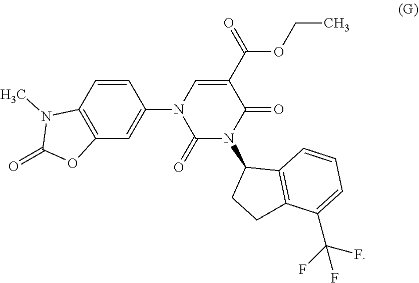 [SMILES: CCOC(=O)c1cn(-c2ccc3c(c2)oc(=O)n3C)c(=O)n([C@@H]2CCc3c2cccc3C(F)(F)F)c1=O]

8. The combination of claim 7, wherein the chymase inhibitor is 1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (C)

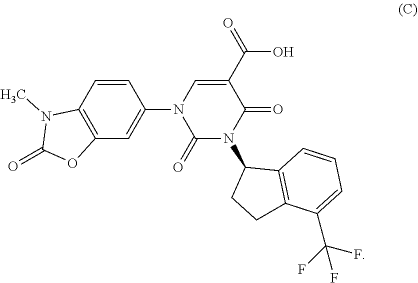 [SMILES: Cn1c(=O)oc2cc(-n3cc(C(=O)O)c(=O)n([C@@H]4CCc5c4cccc5C(F)(F)F)c3=O)ccc21]

9. A combination comprising [4-(3,4-Dihydroisoquinolin-2(1H)-yl)piperidin-1-yl][2-(2-oxa-6-azaspiro[3.3]hept-6-yl)pyrimidin-5-yl]methanone and 1-(3-methyl-2-oxo-2,3-dihydro-1,3-benzoxazol-6-yl)-2,4-dioxo-3-[(1R)-4-(trifluoromethyl)-2,3-dihydro-1H-inden-1-yl]-1,2,3,4-tetrahydropyrimidine-5-carboxylic acid (R enantiomer) of formula (C).

10. A method for treatment and/or prophylaxis of peripheral artery diseases (PAD) and/or critical limb ischemia (CLI) comprising administering to a patient in need thereof an effective amount of the combination according to claim 7.

11. A method for treatment and/or prophylaxis of critical limb ischemia (CLI) comprising administering to a patient in need thereof an effective amount of the combination according to claim 7.

12. A method for treatment and/or prophylaxis of peripheral artery diseases (PAD) and/or critical limb ischemia (CLI) comprising administering to a patient in need thereof an effective amount of a pharmaceutical formulation comprising at least one combination according to claim 7.

13-16. (canceled)

17. The method of claim 6, wherein
R¹ is C₂-C₄-alkyl,
where alkyl is substituted by a substituent selected from the group consisting of hydroxy and methoxy,
and
R² is hydrogen,
or
R¹ and R² together with the nitrogen atom to which they are attached form an azetidine, pyrrolidine, morpholine, or 1,1-dioxidothiomorpholine,
where azetidine, pyrrolidine, morpholine, or 1,1-dioxidothiomorpholine may be substituted by 1 to 2 substituents selected independently from the group consisting of hydroxycarbonyl, methyl, trifluoromethyl, methoxy, and methoxymethyl,
or
R¹ and R² together with the nitrogen atom to which they are attached form an azetidine,
where the azetidine may have two substituents which, together with the carbon atom of the azetidine to which they are jointly attached, form an oxetane or 1,1-dioxidothietane,
R³ is hydrogen, fluorine, or methoxy,
and
R⁴ is hydrogen,
or
R³ is hydrogen,
and
R⁴ is hydrogen, fluorine, or methoxy.

18. The method of claim 6, wherein
R¹ is C₂-C₆-alkyl,
where alkyl is substituted by a substituent selected from the group consisting of hydroxy, methoxy, and ethoxy,
and
R² is hydrogen,
or
R¹ and R² together with the nitrogen atom to which they are attached form an azetidine, pyrrolidine, piperidine, azepane, piperazine, morpholine, thiomorpholine, 1-oxidothiomorpholine, or 1,1-dioxidothiomorpholine,
where azetidine, pyrrolidine, piperidine, azepane, piperazine, morpholine, thiomorpholine, 1-oxidothiomorpholine, and 1,1-dioxidothiomorpholine may be substituted by 1 to 2 substituents independently of one another selected from the group consisting of hydroxy, hydroxycarbonyl, C₁-C₃-alkyl, and methoxy,
or
where azetidine, pyrrolidine, piperidine, azepane, piperazine, and morpholine may have two substituents which, together with the carbon atom of the azetidine, pyrrolidine, piperidine, azepane, piperazine, or morpholine to which they are jointly attached, form an azetidine or oxetane,
where this azetidine or oxetane for its part may be substituted by 1 to 2 substituents independently of one another selected from the group consisting of methyl and ethyl,
R³ is hydrogen,
and
R⁴ is hydrogen, fluorine, or methoxy,
or
R³ is hydrogen, fluorine, or methoxy,
and
R⁴ is hydrogen.

19. The method of claim 6, wherein the selective adrenoreceptor α_{2C} receptor antagonist is [4-(3,4-Dihydroisoquinolin-2(1H)-yl)piperidin-1-yl][2-(2-oxa-6-azaspiro[3.3]hept-6-yl)pyrimidin-5-yl]methanone of formula

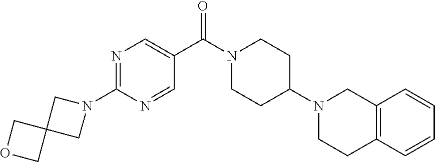 [SMILES: O=C(c1cnc(N2CC3(COC3)C2)nc1)N1CCC(N2CCc3ccccc3C2)CC1]

20. A method for treatment and/or prophylaxis of peripheral artery diseases (PAD) and/or critical limb ischemia (CLI) comprising administering to a patient in need thereof an effective amount of the combination according to claim 8.

21. A method for treatment and/or prophylaxis of critical limb ischemia (CLI) comprising administering to a patient in need thereof an effective amount of the combination according to claim 8.

22. A method for treatment and/or prophylaxis of peripheral artery diseases (PAD) and/or critical limb ischemia (CLI) comprising administering to a patient in need thereof an effective amount of a pharmaceutical formulation comprising at least one combination according to claim 8.

23. A method for treatment and/or prophylaxis of peripheral artery diseases (PAD) and/or critical limb ischemia (CLI) comprising administering to a patient in need thereof an effective amount of the combination according to claim 9.

24. A method for treatment and/or prophylaxis of critical limb ischemia (CLI) comprising administering to a patient in need thereof an effective amount of the combination according to claim 9.

25. A method for treatment and/or prophylaxis of peripheral artery diseases (PAD) and/or critical limb ischemia (CLI) comprising administering to a patient in need thereof an effective amount of a pharmaceutical formulation comprising at least one combination according to claim 9.
